(19) United States
(12) Patent Application Publication
(10) Pub. No.: US 2025/0137031 A1
(43) Pub. Date: May 1, 2025

(54) ASSAY DEVICE

(71) Applicant: CERNERETEC LTD, Bromborough (GB)

(72) Inventors: Jake HODGSON, Chester (GB); Philip BAINES, Wirral (GB)

(21) Appl. No.: 18/834,065

(22) PCT Filed: Mar. 27, 2023
(86) PCT No.: PCT/GB2023/050787

§ 371 Date: Jul. 29, 2024

(30) Foreign Application Priority Data
Apr. 1, 2022 (GB) 2204773.2

(51) Int. Cl. C12Q 1/34; B01J 27/055; C12N 9/10; C12N 9/80; C12Q 1/48

(52) U.S. Cl. CPC C12Q 1/34; B01J 27/055; C12N 9/1096; C12N 9/80; C12Q 1/48; C12Y 206/01002; C12Y 305/01013

(57) ABSTRACT

An assay device for use with samples of biological fluid, the assay device comprising: an N-acetyl-p-aminophenol (APAP) detection unit for detecting and/or measuring APAP in a biological fluid sample; and an alanine aminotransaminase 1 (ALT1) detection unit for detecting and/or measuring ALT1 in said biological fluid sample, wherein the APAP detection unit and the ALT1 detection unit act together as part of an assay process.

### FIELD

The present invention relates to an assay device and method useful with samples of biological fluid, in particular for diagnosing and/or indicating N-acetyl-p-aminophenol overdose.

### BACKGROUND

N-acetyl-p-aminophenol (APAP), also known as paracetamol or acetaminophen, is a widely available painkilling drug. While generally safe at low doses, overdoses of APAP can lead to severe liver damage and can be fatal. Overdoses may result if a user accidentally takes APAP more frequently and/or at larger doses than indicated. In some cases, overdoses are a deliberate attempt at self-harm.

Overdoses or suspected overdoses of APAP are common in hospital emergency rooms. The correct treatment depends on the circumstances, specifically how much APAP has been ingested and how long since the ingestion occurred. Typically, the blood concentration of APAP in a patient is determined by taking a blood sample from the patient and analysing the sample in a hospital laboratory. However, the current practice requires the patient to wait up to four hours in the emergency room before the sample is taken, followed by up to two hours for the sample to be analysed and the results returned. Furthermore, patients must indicate when or how much APAP they have ingested. If the blood concentration of APAP and the time of ingestion and/or dose are known, practitioners can use a Rumack-Matthew nomogram to guide treatment, in particular whether or not to give acetylcysteine treatment. However, the Rumack-Matthew nomogram cannot be used for patients with repeated ingestion of APAP or if the time of ingestion and dose is unknown, for example if the patient is unwilling or unable to indicate when and how much APAP they have taken. APAP blood concentration alone does not indicate the time of ingestion because APAP is metabolised by the body. The rate at which APAP is metabolised by the body differs on a patient by patient basis and may be affected by complicating factors such as age and other drugs ingested at the same time. Therefore, it is not possible to tell if a patient has taken a large dose a longer time ago or a small dose more recently.

There is, therefore, a need for a simple, efficient test which can be used to diagnose an APAP overdose in a patient without needing to know the time or amount of APAP ingestion. It would be particularly advantageous to provide a test which can be used to provide results in a timely manner in the clinical setting, i.e. within a few minutes of a biological sample being provided by the patient.

### SUMMARY

It is one aim of the present invention, amongst others, to provide an assay device which can be used to diagnose an N-acetyl-p-aminophenol (APAP) overdose in a patient. Another aim of the invention is to provide such a device which is simple and efficient to use, and which can provide a substantially immediate result in a clinical setting.

The present invention provides an assay device and a method of diagnosing an APAP overdose as set forth in the appended claims. Other features of the invention will be apparent from the dependent claims, and the description, which follows.

According to a first aspect of the present invention, there is provided an assay device for use with samples of biological fluid, the assay device comprising:
an N-acetyl-p-aminophenol (APAP) detection unit for detecting and/or measuring APAP in a biological fluid sample; and
an alanine aminotransaminase 1 (ALT1) detection unit for detecting and/or measuring ALT1 in said biological fluid sample, wherein the APAP detection unit and the ALT1 detection unit act together as part of an assay process.

According to a second aspect of the present invention, there is provided a method of diagnosing an N-acetyl-p-aminophenol (APAP) overdose, the method comprising the steps of:
introducing a biological fluid sample obtained from a patient into an assay device, wherein the assay device comprises an APAP detection unit and an alanine aminotransaminase 1 (ALT1) detection unit;
detecting and/or measuring APAP in the biological fluid sample and detecting and/or measuring ALT1 in the biological fluid sample,
wherein the APAP and ALT1 measurements are indicative of the ingestion time and size of an APAP dose.

The assay device and method of the invention may be used to diagnose an APAP overdose. APAP levels in the blood rise quickly after ingestion and fall more slowly as it is metabolised. Excess APAP in the body causes damage to the liver. ALT1 is a liver damage marker. Therefore, ALT1 levels will rise progressively during the time excess APAP is causing liver damage. Using APAP and ALT1 levels together the clinician can assess if the dose is recent and non-critical (low ALT1, low APAP), recent and critical (low ALT1, high APAP), or less recent and critical (high ALT1, low APAP). This assessment informs the most appropriate course of treatment, even if the time of ingestion is not known, because the level of ALT1 is indicative of the recency of the APAP overdose.

The assay device and method of the invention typically provide results in a few minutes, which may be critical in the treatment of an overdose. The assay device and method of the invention are able to provide such timely results because they are able to act upon a biological fluid directly obtained from a patient (i.e. without the need for any prior processing of the biological fluid sample) and/or because they detect and/or measure APAP and ALT1 in the biological fluid at the same time, which provides an accurate result without the need for further tests. Furthermore, the assay device and method of the invention may be used for repeated testing of a patient to help determine the most appropriate course of treatment.

ALT1 is believed to advantageously be a more specific marker for liver damage caused by APAP overdose compared to other liver damage markers such as aspartate aminotransferase (AST) or γ-glutamyl transferase (GGT).

Throughout this specification, the term “comprising” or “comprises” means including the component(s) specified but not to the exclusion of the presence of other components.

The optional features set out herein may be used either individually or in combination with each other where appropriate and particularly in the combinations as set out in the accompanying claims. The optional features for each exemplary embodiment of the invention, as set out herein are also applicable to any other aspects or exemplary embodiments of the invention, where appropriate. In other words, the skilled person reading this specification should consider the optional features for each aspect or embodiment of the invention as interchangeable and combinable between different aspects of the invention.

The first aspect of the invention provides an assay device. The term “assay device” is used herein in relation to all aspects of the invention to mean a device that conducts an assay, i.e. which conducts an investigative, analytical procedure so as to qualitatively assess and/or measure one or more properties of a biological fluid sample.

The assay device of the first aspect of the invention is for use with a sample of biological fluid. The term “biological fluid” is used herein in relation to all aspects of the invention to mean a fluid obtained from a human or animal, particularly from a living human or animal, and includes the fluid directly obtained from the human or animal and the fluid after modification or treatment. Modification or treatment may be by any suitable method, such as by filtering and/or by dilution with or dissolving in an appropriate solvent, for example with or in a suitable buffer solution. Examples of biological fluids include whole blood, plasma, serum, saliva, expectorate and nasal sample, which may be used as obtained from the human or animal body, or which may undergo modification or treatment as discussed herein. Preferably, the sample of biological fluid is whole blood, such as whole blood that has undergone modification or treatment as discussed herein.

For example, the sample may comprise whole blood diluted with or dissolved in an appropriate solvent such as a suitable buffer solution.

Typically, a sample of less than 500 μl is required for use in the assay device and method of the invention. For example, a sample of from 200 to 500 μl may be used. The assay device is, for example, suitable for use with a blood sample obtained by means of a finger or heel prick procedure, wherein the blood sample may undergo modification or treatment as discussed herein. The reference to the sample volume is intended to refer to the volume of the sample which is input into the assay device (such that the sample volume may represent the volume of a sample comprising a biological fluid obtained from the human or animal body and an appropriate solvent). The skilled person would appreciate that the sample volume may decrease if the sample undergoes a pre-treatment step, such as filtering. The ability to conduct the test using small volumes of biological samples makes the test easy to conduct and more comfortable for the patient.

The APAP and/or ALT1 detection and/or measurement may be achieved by conducting optical absorbance measurements at a specified wavelength or at a range of wavelengths. The skilled person would readily determine the suitable wavelength(s) according to the reagent(s) used. For example, the specified wavelength may be 615 nm, which corresponds to the peak absorption of the indophenol dye formed when 2-methylphenol (o-cresol) couples to p-aminophenol.

The APAP detection unit and the ALT1 detection unit act together as part of an assay process. In other words, in the assay device of the first aspect of the invention, the same biological fluid sample is analysed in both the APAP detection unit and the ALT1 detection unit at substantially the same time. Typically, one portion of said biological fluid sample enters the APAP detection unit and another portion of said biological fluid sample enters the ALT1 detection unit. This means that both of the units provide results which are used in the diagnosis of an APAP overdose for a patient. This offers the advantages of a quick and efficient test which enables a physician to act very quickly to treat a patient if the test results indicate a problem. Analysing the same biological fluid sample in both the APAP detection unit and the ALT1 detection unit also ensures that the APAP detection and/or measurement and the ALT1 detection and/or measurement correspond to a single point in time for the patient. Thus, the assay device of the invention may improve the accuracy of a diagnosis of an APAP overdose.

The assay device of the first aspect of the invention may further comprise a fluid introducing portion. The assay device may further comprise a filter located at or near to the fluid introducing portion. In other words, the assay device may comprise an integrated filter. The filter, when present, acts to filter a biological fluid sample in a pre-treatment step. After passing through the filter in the pre-treatment step, said biological fluid sample passes through the APAP detection unit and the ALT1 detection unit.

As the skilled person would appreciate, any suitable filter may be used. Examples of suitable filters include plasma filters such as a Fusion 5 filter from G E Healthcare, a Vivid filter from Pall Corporation, a CytoSep 1667 filter from Ahlstrom-Munksjo and a Cobetter separation membrane. For example, when a biological fluid sample is whole blood the filter separates plasma from the whole blood, such that the plasma then enters the APAP detection and ALT1 detection units for analysis. The use of a filter in the assay device means that it is unnecessary to process said biological fluid sample prior to its testing in the assay device. This reduces the time required to provide an output and enables diagnosis in the patient setting. Thus, the assay device of the invention can be used in a single step method as described herein. The use of an integrated filter in the assay device also enables the device to function with small sample volumes, for example volumes obtained by a finger or heel prick method. Volumes such as these could be significantly reduced if the sample needed prior processing in a device or method external to the assay device.

The assay device suitably comprises a reservoir located at or near to a fluid introducing portion, preferably located adjacent to a filter when present. In use, the reservoir receives a sample of biological fluid of sufficient volume to flow into each of the APAP and ALT1 detection units. Preferably, the reservoir is located such that, in use, said sample of biological fluid flows through the filter into the reservoir such that said biological fluid collects in the reservoir in a volume sufficient to flow into each of the APAP and ALT1 detection units. The reservoir suitably comprises a sorbent pad, preferably an absorbent pad. The reservoir and the filter may be integrated, such that the reservoir also functions as the filter.

The reservoir may comprise a mechanical barrier on the surface thereof so as to split the reservoir into a first zone and a second zone. In use, the first zone receives the sample of biological fluid and the second zone subsequently receives chase buffer. The chase buffer is a diluent which flows through the first zone and helps to carry the sample of biological fluid into each of the APAP and ALT1 detection units. The first zone is suitably arranged in between the second zone and the APAP and ALT1 detection units. The mechanical barrier prevents the chase buffer from mixing with an unfiltered sample of biological fluid.

Alternatively, the assay device may comprise a chase buffer receiving area which is separate, but in fluid communication with, the reservoir. In such a configuration, no mechanical barrier is required on the surface of the reservoir. The chase buffer receiving area suitably comprises a sorbent pad, preferably an absorbent pad. The sorbent pad suitably comprises a material which causes the chase buffer to quickly flow through the reservoir. The sorbent pad suitably comprises glass fibres. The chase buffer receiving area may comprise a Whatman Fusion 5 pad.

Suitably, the APAP detection unit and/or the ALT1 detection unit is in fluid communication with the fluid introducing portion. When a biological fluid sample is added to the fluid-introducing portion, it is suitably able to pass into the APAP detection unit and/or the ALT1 detection unit without further input from the user or with addition of a chase buffer. Preferably, both the APAP detection unit and the ALT1 detection unit are in fluid communication with the fluid introducing portion. The fluid introducing portion, the APAP detection unit and the ALT1 detection unit are suitably arranged to allow a biological fluid sample to flow through the assay device by capillary action (i.e. wicking).

The assay device may comprise a reference unit or channel which acts as a baseline. The reference unit or channel houses a portion of a biological fluid sample prior to its entry into the APAP and/or ALT1 detection unit for comparison with the sample after the APAP and/or ALT1 assay has been conducted. For example, when the APAP detection unit conducts an assay which makes colorimetric measurements, the reference unit or channel provides a baseline colour with which to compare after the assay has been conducted.

Suitably, the APAP detection unit and/or the ALT1 detection unit comprises a channel in which a biological assay is conducted. Preferably the APAP detection unit comprises a channel in which an APAP detection assay is conducted and the ALT1 detection unit comprises a channel in which an ALT1 detection assay is conducted. The channel is suitably arranged such that a biological fluid sample enters one end of the channel and travels along the channel, thereby undergoing a biological assay. Suitably, the channel has a width of from 1 to 2.5 mm, such as from 1.5 to 2.3 mm, for example 2 mm. Channel widths within this range have been found to provide an ideal flow rate of the sample along the channel. Channel widths of 3 mm or greater may disadvantageously reduce the capillary action and therefore the flow speed of the sample,

The channel may comprise a sorbent pad. By sorbent pad, we mean a piece of material which is absorbent or adsorbent for biological fluids. Suitably, the sorbent pad permits a biological fluid to move through the sorbent pad, preferably through capillary action (i.e. wicking). The sorbent pad may comprise a permeable membrane. Suitably, the device comprises a backing on which the sorbent pad is arranged. The backing is suitably on the opposite side of the sorbent pad to the fluid introducing portion of the device. Suitably, the sorbent pad is in the form of a strip or a sheet.

Suitably, the channel comprises a plurality of sorbent pads. Suitably, each sorbent pad is in the form of a strip or a sheet. Suitably, each sorbent pad is in contact with at least one other of the sorbent pads. Preferably, each sorbent pad overlaps (preferably lengthways) with at least one other of the sorbent pads. This allows a biological fluid sample to flow from one pad to another, preferably through capillary action. Each sorbent pad suitably has the same width as the channel. Suitably, each sorbent pad independently has a length of at least 6 mm, such as at least 8 mm. Suitably, the length of the overlap between the sorbent pads is independently at least 2.5 mm, such as at least 3.5 mm, such as 4 mm. It has been found that overlap lengths of 2 mm or less may disadvantageously reduce the flow of the sample from one pad to another.

The chase buffer receiving area is suitably in contact with the reservoir. Preferably the reservoir comprises a sorbent pad, the chase buffer receiving area comprises a sorbent pad, and the reservoir overlaps with the chase buffer receiving area. In the region of the overlap, preferably the chase buffer receiving area is proximal to (and the reservoir is distal to) the fluid introducing portion. In the region of the overlap, preferably the chase buffer receiving area is distal to (and the reservoir is proximal to) the backing. In the region of the overlap, preferably the chase buffer receiving area is not in contact with the backing and the reservoir is in contact with the backing.

The assay device of the first aspect of the present invention comprises an APAP detection unit. The APAP detection unit suitably comprises a reagent which changes colour in the presence of APAP or a derivative thereof. The reagent is suitably colourless. The reagent suitably comprises an aromatic or heteroaromatic compound.

Suitably, the APAP detection unit comprises a first reagent which is operative to react with APAP to form an APAP derivative and a second reagent which is operative to react with the derivative to form a coloured product.

The first reagent is suitably operative to hydrolyse APAP to form p-aminophenol. The first reagent suitably comprises an enzyme, such as an aryl acyl amidase.

The second reagent is suitably operative to react with p-aminophenol to form a coloured product, such as an indophenol dye. Suitably, the second reagent comprises a hydroxyl-substituted aromatic or heteroaromatic compound. By “aromatic compound” we mean a compound containing one or more aromatic rings wherein the ring atoms are carbon atoms. By “heteroaromatic compound” we mean a compound containing one or more aromatic rings, wherein at least one aromatic ring contains one or more heteroatoms in the ring. By “heteroatom” we mean an atom other than carbon. Suitable heteroatoms include nitrogen, oxygen and sulphur.

The hydroxyl-substituted aromatic compound may comprise a hydrocarbyl-substituted phenol, such as an alkyl-substituted phenol. The alkyl-substituted phenol may comprise a phenol substituted with one or more methyl groups. The hydroxyl-substituted heteroaromatic compound may comprise a hydroxyl-substituted quinoline. Examples of suitable second reagents include 2-methylphenol (o-cresol), 2,3-dimethylphenol, 2,5-dimethylphenol, 2,6-dimethylphenol, and 8-hydroxyquinoline. Preferably, the second reagent comprises 2-methylphenol.

The second reagent is suitably mobilisable in a biological fluid sample. By “mobilisable” we mean that the reagent is not immobilised and can be carried by a biological fluid sample moving through the device.

The APAP detection unit may comprise a catalyst which is operative to catalyse the reaction between the APAP derivative and the second reagent. The catalyst is suitably operative to catalyse the reaction between p-aminophenol and the second reagent. The catalyst suitably comprises a metal salt, such as a sodium, potassium, chromium, manganese, iron, cobalt, nickel or copper salt. Examples of suitable catalysts include copper sulfate, manganese chloride, and sodium periodate. Copper sulfate is preferred. The copper sulfate may be in a hydrated form, such as copper sulfate pentahydrate.

The APAP detection unit may comprise a pH modifier. The pH modifier may advantageously increase or decrease the pH to a level at which the coloured product is easily detectable. For example, the coloured product may exhibit the strongest single-wavelength absorption in a specific pH range. The pH modifier may also advantageously increase the rate of the reaction between the APAP derivative and the second reagent. Suitably the pH modifier is operative to increase the pH, such as to at least 7, such as at least 8, for example at least 10. The pH modifier may be operative to increase the pH to be within the range of from 7 to 14, such as from 8 to 12, for example from 10 to 11. The pH modifier suitably comprises an ammonium salt, such as an ammonium halide, or a carbonate salt, such as sodium carbonate. Ammonium chloride is preferred.

Suitably, the APAP detection unit comprises a catalyst and a pH modifier as defined herein. Preferably, the APAP detection unit comprises copper sulfate and ammonium chloride.

The APAP detection unit may comprise a first reagent area comprising the first reagent. The first reagent area may comprise a sorbent pad. The first reagent area may be prepared by applying (e.g. spraying) a solution containing the first reagent to a sorbent pad and drying the sorbent pad. The sorbent pad is suitably quick-drying. The sorbent pad suitably comprises glass fibres. The first reagent area may comprise a Whatman Standard 17 pad.

The APAP detection unit may comprise a second reagent area comprising the second reagent. The second reagent area may comprise a sorbent pad. The second reagent area may be prepared by applying (e.g. spraying) a solution containing the second reagent to a sorbent pad and drying the sorbent pad. The sorbent pad suitably comprises a material which causes the biological fluid sample to quickly flow through the second reagent area. The second reagent area may comprise a Whatman Grade 1 pad.

The APAP detection unit may comprise a reaction area comprising the catalyst and/or the pH modifier. The reaction area may comprise a sorbent pad. The reaction area may be prepared by applying (e.g. spraying) a solution containing the catalyst and/or the pH modifier to a sorbent pad and drying the sorbent pad. The sorbent pad suitably comprises a material which causes the biological fluid sample to quickly flow through the reaction area. The reaction area may comprise a Whatman Grade 1 pad.

The APAP detection unit may comprise a wicking pad. The wicking pad is a sorbent pad, such as an absorbent pad. The wicking pad suitably comprises a material on the coloured product is clearly visible, preferably even at low concentrations of the coloured product. The wicking pad suitably comprises glass fibres or cellulose fibres. The wicking pad may comprise a Whatman Standard 17 pad or a Millipore cellulose fibre pad C083.

Suitably, the APAP detection unit comprises a plurality of sorbent pads. Suitably, each sorbent pad is in the form of a strip or a sheet. Suitably, each sorbent pad is in contact with at least one other of the sorbent pads. Preferably, each sorbent pad overlaps (preferably lengthways) with at least one other of the sorbent pads. Suitably, each sorbent pad independently has a width of from 1 to 2.5 mm, such as from 1.5 to 2.3 mm, for example 2 mm. Suitably, each sorbent pad independently has a length of at least 6 mm, such as at least 8 mm. Suitably, the length of the overlap between the sorbent pads is independently at least 2.5 mm, such as at least 3.5 mm, such as 4 mm.

The first reagent area is suitably in contact with the reservoir. Preferably the reservoir comprises a sorbent pad, the first reagent area comprises a sorbent pad, and the reservoir overlaps with the first reagent area. In the region of the overlap, preferably the first reagent area is distal to (and the reservoir is proximal to) the side of device comprising the fluid introducing portion. In the region of the overlap, preferably the first reagent area is proximal to (and the reservoir is distal to) the backing. In the region of the overlap, preferably the first reagent area is in contact with the backing and the reservoir is not in contact with the backing.

The second reagent area is suitably in contact with the first reagent area. Preferably the first reagent area comprises a sorbent pad, the second reagent area comprises a sorbent pad, and the first reagent area overlaps with the second reagent area. In the region of the overlap, preferably the second reagent area is distal to (and the first reagent area is proximal to) the side of device comprising the fluid introducing portion. In the region of the overlap, preferably the second reagent area is proximal to (and the first reagent area is distal to) the backing. In the region of the overlap, preferably the second reagent area is in contact with the backing and the first reagent area is not in contact with the backing.

The reaction area is suitably in contact with the second reagent area. Preferably the second reagent area comprises a sorbent pad, the reaction area comprises a sorbent pad, and the second reagent area overlaps with the reaction area. In the region of the overlap, preferably the reaction area is distal to (and the second reagent area is proximal to) the side of device comprising the fluid introducing portion. In the region of the overlap, preferably the reaction area is proximal to (and the second reagent area is distal to) the backing. In the region of the overlap, preferably the reaction area is in contact with the backing and the second reagent area is not in contact with the backing.

The wicking pad is suitably in contact with the reaction area. Preferably the reaction area comprises a sorbent pad and the reaction area overlaps with the wicking pad. In the region of the overlap, preferably the wicking pad is proximal to (and the reaction area is distal to) the side of device comprising the fluid introducing portion. In the region of the overlap, preferably the wicking pad is distal to (and the reaction area is proximal to) the backing. In the region of the overlap, preferably wicking pad is not in contact with the backing and the reaction area is in contact with the backing.

The direction of the overlaps described above has been found to improve the flow of the sample through the APAP detection unit.

The APAP detection unit may comprise a read area in contact with the reaction area. The read area is suitably positioned in between the reaction area and the wicking pad. The read area may comprise a sorbent pad and overlap with the reaction area comprising a sorbent pad. The read area may comprise a sorbent pad and overlap with the reaction area comprising a sorbent pad and with the wicking pad. Alternatively, and preferably, the wicking pad may function as a read area. In such an embodiment a separate read area is not required.

The APAP detection unit may comprise a first reagent area, a second reagent area, and a reaction area, wherein the first reagent area comprises a first reagent which is operative to hydrolyse APAP to form p-aminophenol, the second reagent area comprises a second reagent which is operative to react with p-aminophenol to form a coloured product, and the reaction area comprises a catalyst which is operative to catalyse the reaction between p-aminophenol and the second reagent.

The APAP detection unit may comprise a first reagent area, a second reagent area, and a reaction area, wherein the first reagent area comprises a first reagent which is operative to hydrolyse APAP to form p-aminophenol, the second reagent area comprises a second reagent which is operative to react with p-aminophenol to form a coloured product, and the reaction area comprises a pH modifier and a catalyst which is operative to catalyse the reaction between p-aminophenol and the second reagent.

Suitably, the first reagent comprises an enzyme, the second reagent comprises a hydroxyl-substituted aromatic or heteroaromatic compound, and the catalyst comprises a metal salt.

Suitably, the first reagent comprises an enzyme, the second reagent comprises a hydroxyl-substituted aromatic or heteroaromatic compound, the pH modifier comprises an ammonium salt, and the catalyst comprises a metal salt.

Suitably, the first reagent comprises aryl acyl amidase, the second reagent comprises o-cresol, and the catalyst comprises copper sulfate.

Suitably, the first reagent comprises aryl acyl amidase, the second reagent comprises o-cresol, the pH modifier comprises ammonium chloride, and the catalyst comprises copper sulfate.

The assay device of the first aspect of the present invention comprises an ALT1 detection unit. The ALT1 detection unit suitably comprises a conjugate area and a lateral flow test area, wherein the conjugate area comprises an ALT1 binding agent, and the lateral flow test area comprises a test line and a control line.

The conjugate area suitably comprises a sorbent pad. The conjugate area may be prepared by applying (e.g. spraying) a solution containing the ALT1 binding agent to a sorbent pad and drying the sorbent pad. The sorbent pad is suitably quick-drying. The sorbent pad suitably comprises glass fibres. The conjugate area may comprise a Whatman Standard 17 pad.

The ALT1 binding agent suitably comprises an ALT1 binding portion conjugated to a detection species. The ALT1 binding portion preferably comprises an antibody. The antibody is suitably specific to ALT1 and not to ALT2. The detection species is suitably coloured, fluorescent and/or magnetic. Preferably the detection species is a coloured species (i.e. a chromophore). The ALT1 binding reagent is suitably mobilisable in a biological fluid sample.

The conjugate area may comprise a secondary detection agent. The secondary detection agent suitably comprises a binding portion conjugated to a detection species. The binding portion suitably does not bind to ALT1. The detection species is suitably coloured, fluorescent and/or magnetic. Preferably the detection species is a coloured species (i.e. a chromophore). The secondary detection reagent is suitably mobilisable in a biological fluid sample.

The lateral flow test area suitably comprises a sorbent pad. The lateral flow test area may comprise a nitrocellulose membrane, such as Whatman FF120HP.

The test line suitably comprises a test reagent which binds to a complex formed from ALT1 and the ALT1 binding agent. Preferably, the test reagent binds specifically to a complex formed from ALT1 and the ALT1 binding agent. By binding specifically, the test reagent does not bind to other molecules, such as free ALT1 binding agent or the secondary detection agent, if present. The test reagent suitably comprises an antibody.

The test reagent is suitably immobilised. By “immobilised” we mean that the reagent remains fixed in place and is not carried by a biological fluid sample moving through the device. This allows the complex carried by said biological fluid sample to accumulate at the test line. Thereby the complex can be detected, preferably by the appearance of a coloured line.

The control line comprises a control reagent which binds to the ALT1 binding agent and/or to the secondary detection agent, if present. The control reagent may comprise an antibody. The control reagent is suitably immobilised. This allows any free ALT1 binding agent and/or secondary detection agent to accumulate at the control line. Thereby the free ALT1 binding agent and/or secondary detection agent can be detected, preferably by the appearance of a coloured line.

When the conjugate area comprises a secondary detection agent, the control reagent preferably binds to the secondary detection agent and does not bind to the ALT1 binding agent. This advantageously allows the control line to be reliably detected regardless of the amount of ALT1 present in the biological fluid sample.

Suitably, the test line comprises a test reagent which binds to a complex formed from ALT1 and the ALT1 binding agent, and the control line comprises a control reagent which binds to the ALT1 binding agent. Alternatively, the test line comprises a test reagent which binds to a complex formed from ALT1 and the ALT1 binding agent, and the control line comprises a control reagent which binds to the secondary detection agent. The test reagent and the control reagent are suitably immobilised.

The test line is preferably closer than the control line to the conjugate area. This ensures that a complex formed from ALT1 and the ALT1 binding agent accumulates at the test line instead of the control line.

The lateral flow test area may be prepared by applying (e.g. spraying) a solution containing the test reagent to form a test line on a sorbent pad and applying (e.g. spraying) a solution containing the control reagent to form a control line on the sorbent pad and drying the sorbent pad.

The ALT1 detection unit may comprise a wicking pad. The wicking pad is a sorbent pad, such as an absorbent pad. The wicking pad may comprise a Whatman Fusion 5 pad.

Suitably, the ALT1 detection unit comprises a plurality of sorbent pads. Suitably, each sorbent pad is in the form of a strip or a sheet. Suitably, each sorbent pad is in contact with at least one other of the sorbent pads. Preferably, each sorbent pad overlaps with at least one other of the sorbent pads. Suitably, each sorbent pad independently has a width of from 3 to 5 mm, such as from 3.5 to 4.5 mm, for example 4 mm. Suitably, each sorbent pad independently has a length of at least 6 mm, such as at least 8 mm. Suitably, the length of the overlap between the sorbent pads is independently at least 2.5 mm, such as at least 3.5 mm, such as 4 mm.

The conjugate area is suitably in contact with the reservoir. Preferably the reservoir comprises a sorbent pad, the conjugate area comprises a sorbent pad, and the reservoir overlaps with the conjugate area. In the region of the overlap, preferably the conjugate area is distal to (and the reservoir is proximal to) the side of device comprising the fluid introducing portion. In the region of the overlap, preferably the conjugate area is proximal to (and the reservoir is distal to) the backing. In the region of the overlap, preferably the conjugate area is in contact with the backing and the reservoir is not in contact with the backing.

The lateral flow test area is suitably in contact with the conjugate area. Preferably the conjugate area comprises a sorbent pad, the lateral flow test area comprises a sorbent pad, and the conjugate area overlaps with the lateral flow test area. In the region of the overlap, preferably the lateral flow test area is distal to (and the conjugate area is proximal to) the side of device comprising the fluid introducing portion. In the region of the overlap, preferably the lateral flow test area is proximal to (and the conjugate area is distal to) the backing. In the region of the overlap, preferably the lateral flow test area is in contact with the backing and the conjugate area is not in contact with the backing.

The wicking pad is suitably in contact with the lateral flow test area. Preferably the lateral flow test area comprises a sorbent pad and the lateral flow test area overlaps with the wicking pad. In the region of the overlap, preferably the wicking pad is proximal to (and the lateral flow test area is distal to) the side of device comprising the fluid introducing portion. In the region of the overlap, preferably the wicking pad is distal to (and the lateral flow test area is proximal to) the backing. In the region of the overlap, preferably wicking pad is not in contact with the backing and the lateral flow test area is in contact with the backing.

The direction of the overlaps described above has been found to improve the flow of the sample through the ALT1 detection unit.

The assay device of the first aspect suitably comprises a housing comprising one or more apertures through which results from the APAP detection unit and/or the ALT1 detection unit are visible. Suitably the apertures are adjacent to the reaction area or read area (if present) of the APAP detection unit and the lateral flow test area of the ALT1 detection unit. The fluid introducing portion is suitably in the form of an aperture in the housing.

The housing is suitably formed from a material which is impermeable to a biological fluid sample. The material is suitably impermeable to water. The material may be a polymeric material.

Suitable polymeric materials include acrylonitrile butadiene styrene (ABS), polycarbonates, and polyamides.

The assay device of the first aspect of the invention is suitably handheld.

The second aspect of the invention provides a method of diagnosing an APAP overdose.

Suitably, the results the APAP and ALT1 detection and/or measurement are indicative of the ingestion time and size of an APAP dose.

The method of the second aspect of the invention may be conducted using any suitable assay device, including but not limited to an assay device according to the first aspect of the invention.

The method of the second aspect is carried out on a biological fluid sample obtained from a patient. The method does not comprise a step of obtaining the biological fluid sample from the patient. The biological fluid sample is not reintroduced into the patient.

The biological fluid sample is suitably blood or plasma. Suitably, the blood is whole blood. By whole blood we mean unprocessed blood which has not had any components removed. Preferably the assay device comprises an integrated filter and the biological fluid sample is whole blood. This simplifies the method by removing the need for pre-processing the biological fluid sample.

The biological fluid sample is suitably introduced into the assay device via a fluid introducing portion of the device.

The method of the second aspect may further comprise the step of introducing a chase buffer into the assay device following introduction of the biological fluid sample into the assay device. This helps the biological fluid sample flow through the device. The chase buffer may be introduced into the assay device via a fluid introducing portion of the device.

APAP in the biological fluid sample is suitably detected by a colour change in the APAP detection unit. APAP in the biological fluid sample may be measured by measuring a colour change in the APAP detection unit. For example, the change in absorbance of a single wavelength, such as 615 nm, may be measured. The colour change is suitably caused by reaction of a reagent with APAP or a derivative thereof to form a coloured product, such as an indophenol dye. The amount of APAP in the biological fluid sample may be measured by measuring the colour change caused by the coloured product. The colour change may be measured by use of an optical measurement device, such as an Immunochromato reader from Hamamatsu Photonics. The colour change is suitably detected and/or measured in a reaction area or read area (e.g. the wicking pad) of the APAP detection unit. Suitably the APAP detection unit may detect APAP at concentrations of 10 μg/mL or higher.

ALT1 in the biological fluid sample is suitably detected by a change in colour, fluorescence and/or magnetism in the ALT1 detection unit. ALT1 in the biological fluid sample may be measured by measuring a change in colour, fluorescence and/or magnetism in the ALT1 detection unit. For example, the change in colour and/or fluorescence may be measured by measuring the change in absorbance of a single wavelength. The change in colour, fluorescence and/or magnetism may be measured by use of an optical measurement device, fluorescence measurement device, and/or magnetic measurement device. A suitable optical measurement device and fluorescence measurement device is an Immunochromato reader from Hamamatsu Photonics. The change in colour, fluorescence and/or magnetism is suitably detected and/or measured at a test line of the ALT1 detection unit. The change is typically caused by accumulation of a complex formed from ALT1 and an ALT1 binding agent at the test line, wherein the ALT1 binding agent comprises a detection species which is coloured, fluorescent and/or magnetic. Preferably, ALT1 in the biological fluid sample is detected by a change in colour in the ALT1 detection unit. ALT1 in the biological fluid sample is suitably measured by measuring a colour change in the ALT1 detection unit.

Suitably, APAP in the biological fluid sample is detected by a colour change in the APAP detection unit and/or ALT1 in the biological fluid sample is detected by a change in colour, fluorescence and/or magnetism in the ALT1 detection unit. Preferably, APAP in the biological fluid sample is detected by a colour change in the APAP detection unit and/or ALT1 in the biological fluid sample is detected by a change in colour in the ALT1 detection unit.

According to a third aspect of the present invention, there is provided the use of an assay device according to the first aspect of the invention for diagnosing an N-acetyl-p-aminophenol (APAP) overdose.

### BRIEF DESCRIPTION OF THE DRAWINGS

For a better understanding of the invention, and to show how exemplary embodiments of the same may be carried into effect, reference will be made, by way of example only, to the accompanying diagrammatic Figures, in which:

FIG. 1 shows a schematic view of an assay device according to the first aspect of the invention.

### DESCRIPTION OF EMBODIMENTS

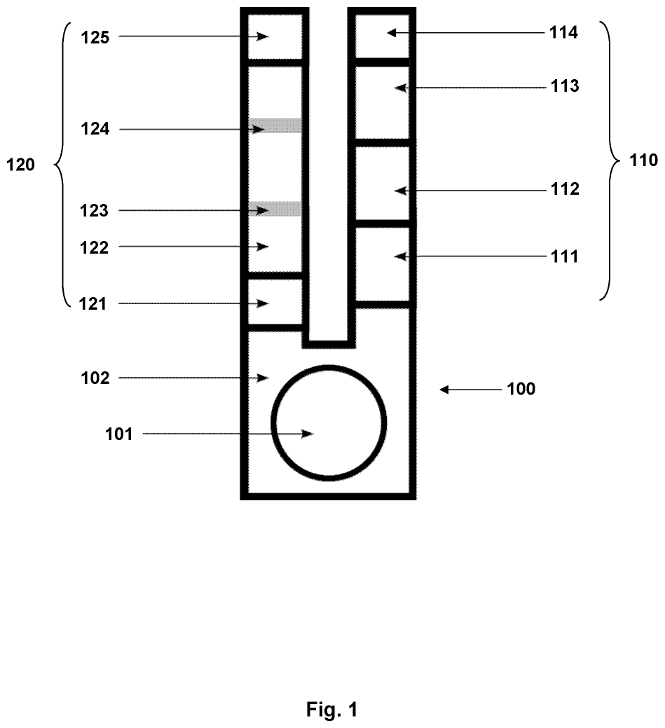

FIG. 1 shows a schematic view of an assay device 100 for use with samples of biological fluid according to an example embodiment of the invention. The assay device comprises an N-acetyl-p-aminophenol (APAP) detection unit 110 and an alanine aminotransaminase 1 (ALT1) detection unit 120.

The APAP detection unit 110 and the ALT1 detection unit 120 are arranged to receive the same biological fluid sample from a patient, which in this example embodiment is whole blood. The whole blood is added onto a blood filter 101 which filters red blood cells and allows plasma to flow through into an absorbent support pad 102.

From the support pad 102, a portion of the blood plasma passes to the APAP detection unit 110. The APAP detection unit 110 is arranged to detect and/or measure APAP in the biological fluid sample. The APAP detection unit 110 comprises a first reagent area 111, a second reagent area 112, a reaction area 113, and a wicking pad 114. The first reagent area 111 comprises a sorbent pad comprising aryl acyl amidase. The plasma passes through the first reagent area 111 and the aryl acyl amidase hydrolyses any APAP in the plasma to form p-aminophenol. The second reagent area 112 comprises a sorbent pad comprising o-cresol. The plasma passes through the second reagent area 112 and picks up the o-cresol. The reaction area 113 comprises a sorbent pad comprising ammonium chloride and copper sulfate pentahydrate. The plasma passes through the reaction area 113 and the copper sulfate catalyses a reaction between the o-cresol and any p-aminophenol present to form an indophenol dye. The wicking pad 114 is formed from an absorbent material. The wicking pad 114 assists the movement of the plasma through the APAP detection unit 110 and absorbs any excess plasma.

The formation of the indophenol dye causes a colour change. The presence of APAP in the plasma can therefore be detected visually. A quantitative measure of the amount of APAP in the plasma can be obtained, if desired, by measuring the change in colour caused by the dye, for example by using an optical measurement device. The wicking pad 114 advantageously accumulates the indophenol dye and is therefore a preferred location for measuring the colour change.

From the support pad 102, another portion of the blood plasma passes to the ALT1 detection unit 120. The ALT1 detection unit 120 is arranged to detect and/or measure ALT1 in the biological fluid sample. The ALT1 detection unit 120 comprises a conjugate area 121, a lateral flow test area 122, and a wicking pad 125. The conjugate area 121 comprises a sorbent pad and a movably supported conjugate, which is an ALT1 antibody conjugated to a chromophore. The plasma passes through the conjugate area 121 and picks up the conjugate. Any ALT1 present in the plasma binds to the chromophore-conjugated ALT1 antibody to form a coloured complex. The lateral flow test area 122 comprises a sorbent pad comprising blocked nitrocellulose on which there is a test line 123 and a control line 124. The test line 123 comprises an immobilised ALT1 capture antibody. The control line 124 comprises an immobilised conjugate-specific capture antibody. When the plasma passes through the lateral flow test area 122, any coloured complex present binds to the test line 123 while free chromophore-conjugated ALT1 antibody binds to the control line 124. The accumulation of coloured species at the test line 123 and the control line 124 provides a visual indication of the presence of ALT1 in the plasma. The wicking pad 125 is formed from an absorbent material. The wicking pad 225 assists the movement of the plasma through the ALT1 detection unit 120 and absorbs any excess plasma.

The ALT1 antibodies used herein are suitably specific to ALT1 and not to ALT2. The ALT1 antibodies may be obtained from synthetic Human Combinatorial Antibody Libraries (HuCAL) by Bio-Rad.

The blood filter 101, support pad 102, APAP detection unit 110 and the ALT1 detection unit 120 are all supported by a backing material (not shown). The support pad 102, first reagent area 111, second reagent area 112, reaction area 113, wicking pad 114, conjugate area 121, lateral flow test area 122, and wicking pad 125 are all formed from separate sorbent pads. The pads are overlapped so that the plasma can wick from one component to the next.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, and drawings), or to any novel one, or any novel combination, of the steps of any method or process so disclosed.

1. An assay device for use with samples of biological fluid, the assay device comprising:
an N-acetyl-p-aminophenol (APAP) detection unit for detecting and/or measuring APAP in a biological fluid sample; and
an alanine aminotransaminase 1 (ALT1) detection unit for detecting and/or measuring ALT1 in said biological fluid sample, wherein the APAP detection unit and the ALT1 detection unit act together as part of an assay process.

2. An assay device according to claim 1, further comprising a fluid introducing portion.

3. An assay device according to claim 2, further comprising a filter located at or near to the fluid introducing portion.

4. An assay device according to claim 2, further comprising a reservoir located at or near to the fluid introducing portion.

5. An assay device according to claim 2, wherein the APAP detection unit and/or the ALT1 detection unit is in fluid communication with the fluid introducing portion.

6. An assay device according to claim 1, wherein the APAP detection unit and/or the ALT1 detection unit comprises a channel in which a biological assay is conducted.

7. An assay device according to claim 6, wherein the channel comprises a plurality of sorbent pads, preferably wherein each sorbent pad overlaps with at least one other of the sorbent pads.

8. An assay device according to claim 1, wherein the APAP detection unit comprises a reagent which changes colour in the presence of APAP or a derivative thereof.

9. An assay device according to claim 1, wherein the APAP detection unit comprises a first reagent area, a second reagent area, and a reaction area, wherein the first reagent area comprises a first reagent which is operative to hydrolyse APAP to form p-aminophenol, the second reagent area comprises a second reagent which is operative to react with p-aminophenol to form a coloured product, and the reaction area comprises a catalyst which is operative to catalyse the reaction between p-aminophenol and the second reagent.

10. An assay device according to claim 9, wherein the first reagent comprises aryl acyl amidase, the second reagent comprises o-cresol, and the catalyst comprises copper sulfate.

11. An assay device according to claim 1, wherein the ALT1 detection unit comprises a conjugate area and a lateral flow test area, wherein the conjugate area comprises an ALT1 binding agent, and the lateral flow test area comprises a test line and a control line.

12. An assay device according to claim 11, wherein the ALT1 binding agent comprises an ALT1 binding portion conjugated to a detection species.

13. An assay device according to claim 12, wherein the ALT1 binding portion comprises an antibody.

14. An assay device according to claim 11, wherein the test line comprises a test reagent which binds to a complex formed from ALT1 and the ALT1 binding agent, and the control line comprises a control reagent which binds to the ALT1 binding agent.

15. An assay device according to claim 1, further comprising a housing comprising one or more apertures through which results from the APAP detection unit and/or the ALT1 detection unit are visible.

16. An assay device according to claim 1, which is handheld.

17. A method of diagnosing an N-acetyl-p-aminophenol (APAP) overdose, the method comprising the steps of:
introducing a biological fluid sample obtained from a patient into an assay device, wherein the assay device comprises an APAP detection unit and an alanine aminotransaminase 1 (ALT1) detection unit;
detecting and/or measuring APAP in the biological fluid sample and detecting and/or measuring ALT1 in the biological fluid sample.

18. A method according to claim 17, wherein the assay device is as defined in any one of claims 1 to 15.

19. A method according to claim 17, wherein the biological fluid sample is whole blood or plasma.

20. A method according to claim 17, wherein APAP in the biological fluid sample is detected by a colour change in the APAP detection unit and/or ALT1 in the biological fluid sample is detected by a change in colour in the ALT1 detection unit.
